(19) United States
(12) Patent Application Publication
(10) Pub. No.: US 2013/0109984 A1
(43) Pub. Date: May 2, 2013

(54) BIOLOGICAL INFORMATION MEASURING BAND WITH A RECHARGEABLE ABILITY

(71) Applicant: Tina Su, Taichung City (TW)

(72) Inventor: Tina Su, Taichung City (TW)

(21) Appl. No.: 13/281,446

(22) Filed: Oct. 26, 2011

(51) Int. Cl. A61B 5/0402

(52) U.S. Cl. 600/509

(57) ABSTRACT

A biological information measuring band with a rechargeable ability includes a signal circuit, a recharge circuit, and at least two measuring points which are electrically connected to the signal circuit. The recharge circuit has a diode and an electrical storage. When the biological information measuring band with a rechargeable ability is recharging, an outer current flows into recharge circuit via the measuring points and the diode allows the outer current to flow into the electrical storage for recharge; but when the biological information measuring band is used by a user, the measuring points receive a heart beat data from the user, and the diode forbids the heart beat data flowing into the recharge circuit such that the heart beat data is transmitted into the signal circuit via the amplifier to display.

### BACKGROUND OF THE INVENTION

1. Field of the Invention

The present invention relates to a biological information measuring device, more particularly to a biological information measuring band with a rechargeable ability.

2. Description of Related Art

Electrocardiography is a transthoracic interpretation of the electrical activity of the heart over a period of time, as detected by electrodes attached to the outer surface of the skin and recorded by a device external to the body. The recording produced by this noninvasive procedure is termed an electrocardiogram. The electrocardiograms can provide a lot of information about humans' health. Thus, there are a plenty of portable biological information measuring devices for people to exam their health today.

The conventional biological information measuring device includes a plurality of electrode-pads, a plurality of wires, and a computer for analysis. The electrode-pads are attached to the skin. The wires connect the electrode-pads to the computer for transmitting the heart beat data to the computer. Finally, the computer analyzes the heart beat data and diagnoses the human's health. However, the conventional biological information measuring device cannot recharge the batteries which lead to a waste of batteries. It is not eco-friendly.

The present invention has arisen to mitigate and/or obviate the disadvantages of the conventional.

### SUMMARY OF THE INVENTION

The main objective of the present invention is to provide an improved biological information measuring band with a rechargeable ability.

To achieve the objective, a biological information measuring band with a rechargeable ability comprises a signal circuit, a recharge circuit, the recharge circuit parallel connected to the signal circuit, at least two measuring points electrically connected to the signal circuit, the signal circuit further comprising an amplifier, the recharge circuit further comprising a diode and at least one electrical storage, the electrical storage is at least two rechargeable batteries in series connection;

thereby when the biological information measuring band with a rechargeable ability is recharging, an outer current flows into the recharge circuit via the measuring points and the diode allows the outer current to flow into the electrical storage of the recharge circuit for recharge; but when the biological information measuring band with a rechargeable ability is used by a user, the measuring points receive a heart beat data from the user, and the diode forbids the heart beat data flowing into the recharge circuit such that the heart beat data is transmitted into the signal circuit via the amplifier to display.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a biological information measuring band with a rechargeable ability of the present invention;

FIG. 2 is a circuit scheme of the biological information measuring band with a rechargeable ability;

FIG. 3 is a perspective view for showing a user wearing the biological information measuring band with a rechargeable ability.

### DETAILED DESCRIPTION OF THE INVENTION

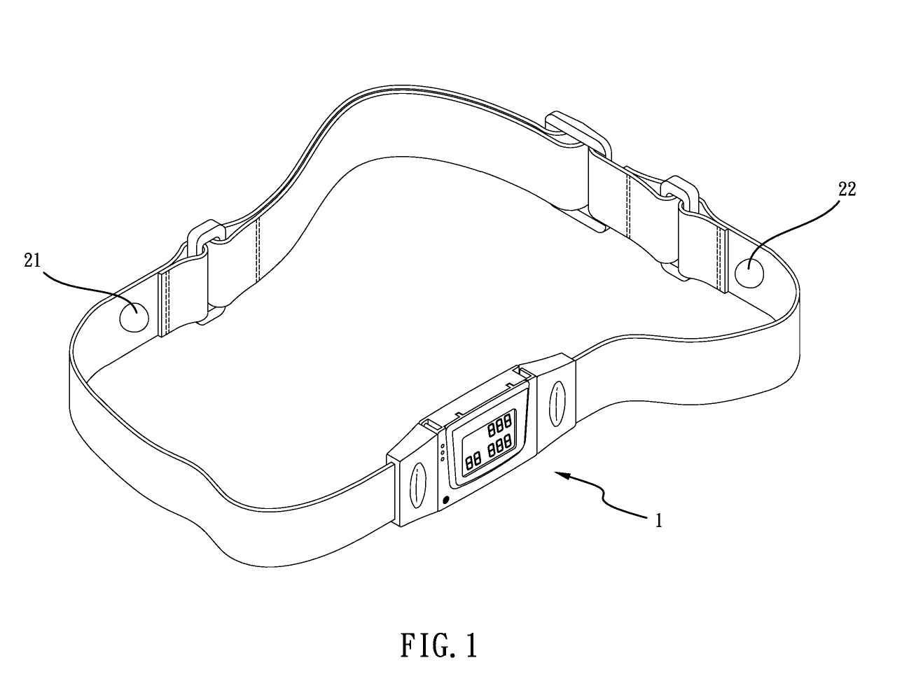

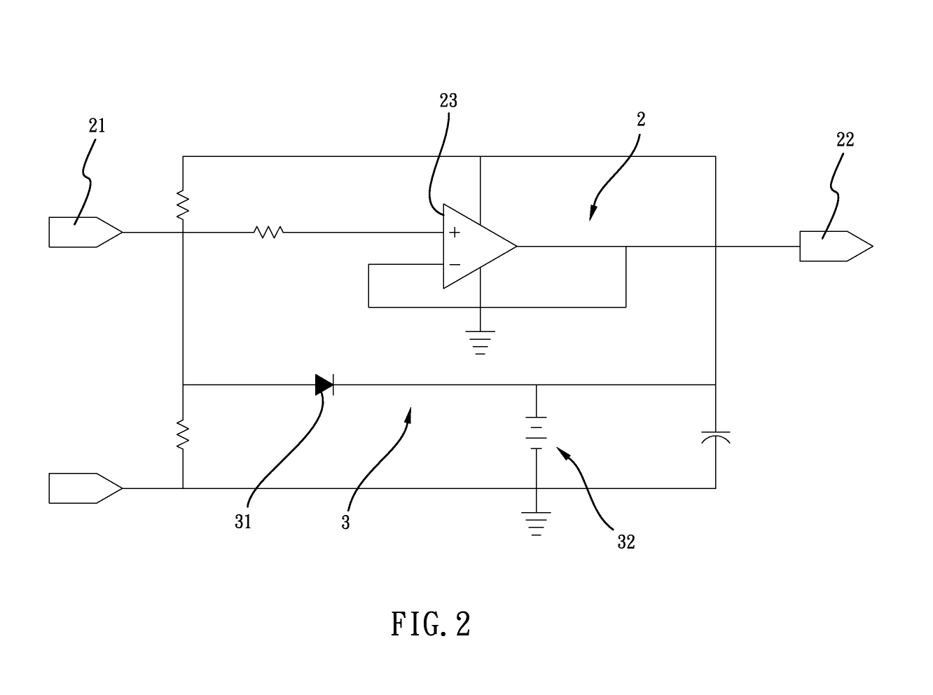

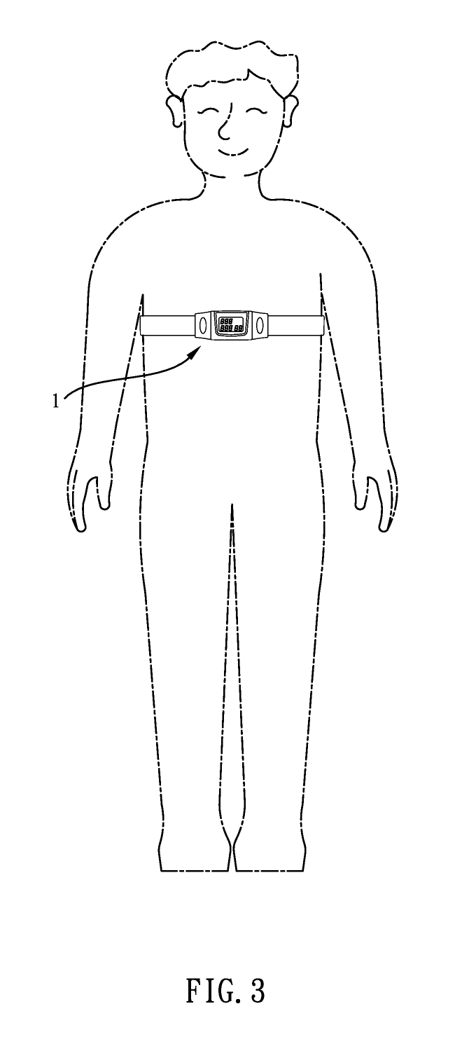

Referring to FIGS. 1-3, a biological information measuring band 1 with a rechargeable ability comprises a signal circuit 2 and a recharge circuit 3. The recharge circuit 3 is parallel connected to the signal circuit 2.

The biological information measuring band 1 has at least two measuring points 21, 22 which are electrically connected to the signal circuit 2. The signal circuit 2 further comprises an amplifier 23. The recharge circuit 3 further comprises a diode 31 and an electrical storage 32 (The electrical storage 32 in the present invention is at least two rechargeable batteries in series connection).

The instruction of the present invention lists as following:

(1) When the biological information measuring band 1 is recharging, an outer current flows into the recharge circuit 3 via the measuring points 21, 22, and the diode 31 allows the outer current to flow into the electrical storage 32 of the recharge circuit 3 for recharge.

(2) When the biological information measuring band 1 is used by a user, the measuring points 21, 22 receive a heart beat data from the user, and the diode 31 forbids the heart beat data flowing into the recharge circuit 3, such that the heart beat data is transmitted into the signal circuit 2 via the amplifier 23 to display.

(3) The diode 31 separates the input signals (the heart beat data or the outer current) from the signal circuit 2 and the recharge circuit 3 for the biological information measuring band 1 to diagnose or to recharge the electrical storage 32.

Therefore, the biological information measuring band 1 with a rechargeable ability has both of the rechargeable function and the diagnostic function. It increases the lifetime of the battery usage and simplifies the structure of the conventional biological information measuring device.

Although the invention has been explained in relation to its preferred embodiment, it is to be understood that many other possible modifications and variations can be made without departing from the spirit and scope of the invention as hereinafter claimed.

What is claimed is:

1. A biological information measuring band with a rechargeable ability comprising:
a signal circuit, a recharge circuit, the recharge circuit parallel connected to the signal circuit, at least two measuring points electrically connected to the signal circuit;
the signal circuit further comprising an amplifier; and
the recharge circuit further comprising a diode and at least one electrical storage;
thereby when the biological information measuring band with a rechargeable ability is recharging, an outer current flows into the recharge circuit via the measuring points and the diode allows the outer current to flow into the electrical storage of the recharge circuit for recharge; but when the biological information measuring band with a rechargeable ability is used by a user, the measuring points receive a heart beat data from the user, and the diode forbids the heart beat data flowing into the recharge circuit such that the heart beat data is transmitted into the signal circuit via the amplifier to display.

2. The biological information measuring band with a rechargeable ability as claimed in claim 1, wherein the electrical storage is at least two rechargeable batteries in series connection.
